(19) United States
(12) Patent Application Publication
(10) Pub. No.: US 2007/0042998 A1
(43) Pub. Date: Feb. 22, 2007

(54) Medical cosmetic method for reducing adipose tissue and for initiating lipolysis through subcutaneous and intracutaneous injections and customized composition compounded from pharmaceutical agents for use therein

(76) Inventor: Annica Karkkainen, Barcelona (ES)

Correspondence Address: RONALD E. GREIGG;GREIGG & GREIGG P.L.L.C., 1423 POWHATAN STREET, UNIT ONE, ALEXANDRIA, VA, 22314, US

(21) Appl. No.: 11/506,202

(22) Filed: Aug. 18, 2006

Related U.S. Application Data
(60) Provisional application No. 60/709,276, filed on Aug. 19, 2005.

(51) Int. Cl. A61K 31/685

(52) U.S. Cl. 514/78

(57) ABSTRACT

The invention describes a method and pharmaceutical compositions for subcutaneous and intracutaneous injection for reduction of localized fat deposits comprising the use of (3-sn-Phosphatidyl)-cholin as a main active and supporting ingredient either on its own, diluted or in combination with compounded agents to initiate the process of lipolysis of the adipose tissue by activating the natural metabolism process in the body. The method can be used on all excess adipose tissue of the body and face, including medical conditions that result from metabolic disorders or those originating from effects from pharmaceutical agents, in addition or apart from lifestyle conditions or other known conditions that generates the growth and expansion of fat cells to accumulate excess body fat. It is the first known effective method of use of such agents in subcutaneous and intracutaneous use that allows a cosmetic reduction of adiposity without surgical intervention. The method of the invention has made it possible to achieve satisfactory cosmetic results in one to four treatment sessions.

### CROSS REFERENCE TO RELATED APPLICATIONS

This application is based on U.S. Provisional Application No. 60/709,276 filed Aug. 19, 2005.

### BACKGROUND OF THE INVENTION

1. Field Of The Invention

The present invention relates to a composition and method for reducing excess adipose tissue, fat cells, on localized fat of the face or body and conditions related thereto, and particularly, to uses of (3-sn-Phosphatidyl)-cholin (PC) lipid enzymes and supporting compounds and analogs thereof to reduce excess body and facial fat and conditions. The effectiveness of the compounds for the intended use lays in their accurate preparation.

2. Description of the Prior Art

The regulation of body weight, and particularly, obesity and conditions related thereto, is a major health concern throughout the world, and particularly in the United States, contributing to morbidity and mortality. Obesity is a metabolic disorder characterized by excessive accumulation of fat stores in adipose tissue. In humans, its causes are a complex interplay of genetics, environment and culture. It is well known that a regimen of diet and exercise leading to weight loss is the best approach for treating obesity, but unfortunately, such regimens are frequently unsuccessful and the individual may seek surgical intervention through various forms of lipoplasty or bariatric surgery. Oftentimes, an individual's inability to lose weight may be due to genetically inherited factors that contribute to increased appetite, a preference for high calorie foods, reduced physical activity and an abnormal metabolism. People inheriting or acquiring such predispositions are prone to obesity regardless of their efforts to combat the condition.

The only known method for reducing fat without diet or exercise has been known to consist of surgical procedures such as liposuction, lipoplasty and/or bariatric surgery. Accordingly, it is a primary objective of the present invention to provide a novel method and means of reducing localized fat deposits of the face and body, is more efficient than previous fat reduction procedures or methods known outside of dieting.

Since 2001 the applicants have been researching the potential of using subcutaneous injections for reduction of localized fat. There has been no prior, stable protocol or understanding of the required biochemical ingredient or preparation of a compound or combination of medical compounds required for effective cosmetic results or a compound approved for cosmetic results without strong discomfort or excess procedures of 5, 8 and even 15 and more treatment sessions.

The applicants assert there is no prior evidence of similar combinations or prior understanding of the required biochemical values of the compounds for the method to create effective medical results. Inaccurate understanding of this aspect to the method will yield no satisfactory results and due to this lack of knowledge, it has been believed that this method is ineffective for cosmetic use. The applicants' method, development and research evidence of the method intended for cosmetic subcutaneous and/or intracutanous proves success and effective cosmetic results from the consequence of our studies and development and is fully unique.

The method of treatment and the unique compounded pharmaceutical agents developed by the applicants have no prior existence as a licensed drug intended for subcutaneous and/or intracutanous use intended for cosmetic procedures to reduce localized fat deposits or metabolic alterations to the adiposity tissue. The pharmaceutical compounds have not existed for prior approved subcutaneous, cosmetic intended use, and not in any published recommended dosage, biochemical structure and combinations that have been proven to be effective for subcutaneous use and the compounds have no prior development for cosmetic use and are unique to the applicants.

Through customized compounding the applicants have managed to develop a formula that has been shown to be comprised of adequate structure and biochemical balance for the medication to be effective and safe in subcutaneous and intracutaneous use—a procedure previously doubted by the medical community.

The applicants have developed a customized compound for this purpose which has been shown to provide less discomfort and improved cosmetic results visible within 1 to 4 treatment sessions.

### OBJECTS OF THE INVENTION

It is an object of the invention to provide a pharmaceutical composition formula that has been shown to be comprised of adequate structure and biochemical balance for the medication to be effective and safe in subcutaneous and intracutaneous use.

It is a further object of the invention to provide a customized compound for this purpose which has been shown to provide less discomfort and improved cosmetic results visible within 1 to 4 treatment sessions.

### SUMMARY OF THE INVENTION

The applicants have investigated and compounded customized lipid preparations that should contain the right biochemical ‘balance’ or mix that can serve as a fat reducing medication that is a relatively safe procedure comparative to the majority of cosmetic procedures and create a visible and proven cosmetic effect for the reduction of adiposity or fat cell or excess fat. This method can be used to reduce any type of excess fat, and need not be restricted to cosmetic effect only, but can also be used in medical conditions such as lipomas, skin retraction, buffalo hump, gynecomastia, pubic fat, and other conditions addressing actual fat tissue.

The method of the invention is effective to create lipolysis, fat necrosis, reducing or ‘melting’ or reduction of fat deposits, the positive evidence of lipolysis taking effect according to the applicants unique discovery of this process. The opposition from medical reports that surgical liposuction is the only effective treatment to address reduction of body fat cells confirm that factual knowledge of this discovery was unknown and uncertain as reliable and effective until the applicants developed and collected evidence on the methods appropriate for the method and pharmaceutical compound in use on human tissue.

One of the most important ingredients of the compounded medications is phosphatidylcholine, particularly soy-based phosphatidylcholine. Phosphatidylcholine makes up the largest choline (lecithin) reservoir in the body and is found in bile. It facilitates the emulsification of fat into the tiniest particles within the nanosphere, enabling the absorption and transportation of fat. After subcutaneous injections of phosphatidylcholine into fat tissue, the adipocytes burst and phosphatidylcholine increases the secretion of triaglycerol-rich lipoproteins. With the help of the lipoproteins and its unique application and the method of claim with the specific process of phosphatidylcholine alone or in combination with compounds, the injected fat and process of lipolysis are transported to the liver to be excreted as bilic acids.

In order to be able to successfully treat cosmetic reduction of face or body fat it is necessary to have a proper understanding of the chemical structure required in the preparation of the 3-sn-Phosphatidylcholin as not all versions of phosphatidylcholine are effective or safe and comfortable for human use. 3-sn-Phosphatidylcholin when prepared properly can be used although not restricted on its own for subcutaneous or intracutanous use for reduction of localized fat although results are enhanced when mixed with other compounds.

The method of the present invention is technically easy and quickly executed, is repeatable and above all harmless in comparison to most cosmetic medical procedures.

The new additions of CLA: Cis-9, trans-11 and Trans-10, cis 12; K2: menaquinone: 2-methyl-3-all-trans-famesyl digeranyl-1, 4-naphthoquinone; K1: Phylloquinone and dihydrophylloquinone are unique optional mixtures to the compound for subcutaneous use. None of the mixtures are a requirement but each provides an enhancement to the recommended main active ingredient 3-sn-Phosphatidylcholin, whether diluted or not.

### DESCRIPTION OF THE PREFERRED EMBODIMENTS

The required compound preparation of lipids is a composition of 70% phosphatidylcholine derived from soy, which uses deoxycholate 4.2% as solvent and benzyl alcohol 3% as preservative. All these ingredients and deoxycholate in particular burst the membranes of the fat cell, while phosphatidylcholine plays the most active part in the lipolysis process. Studies have claimed that injecting deoxycholate alone produced similar effects as this composition claimed with phosphatidylcholine by the applicants. The applicants claim this to only to be true regarding the bursting of the adipocyte membranes, but the subsequent enzymatic reaction that follows and which leads to the actual dissolution of fat is only the result of the biochemical functions of phosphatidylcholine, when it is compounded in a way that encourages lipolysis. This reaction is induced by producing an emulsion of nano-sized monoglycerides that is transported into the liver and metabolized by beta-oxidation, in the citric acid cycle.

The efficacy of the method of the invention has been proven by histopathology images on human tissue with a clear indicating of shrinkage of the fat cells or adipose tissue. In studies with H&E stain treated human fat tissue, the inventive compound and method has shown a unique composition of disrupted fat cells, initiating a clear process of lipolysis that results in the cosmetic and visible reduction of localized fat deposits without surgical intervention. Photo documentation of the efficacy of the developed combination of compounds used in the method exists as a result of studies by the applicants of this method.

Although the cosmetic effect of this discovery has been contradicted in prior studies and others have asserted that surgical lipoplasty is the only proven method until date to reduce localized fat, the applicants have developed a method with proven results based upon our studies of this invention with the unique understanding that the compound should adhere within proper biochemical values to be effective.

The Compound of the Invention

The invention defines a compound for use in a method of decreasing excess fat deposits in a human subject comprising administering to a human subject a lipolysis stimulating compound selected from the group consisting of:

Option 1: (3-sn-Phosphatidyl)-cholin for subcutaneous and/or intracutanous use derived from soya based unsaturated alphalinolic acids, preferred diluted but not restricted to being diluted for subcutaneous use in full, by its own, in part or mixed with other compounds although not restricted to any one. The phosphatidylcholine used for subcutaneous use can contain 70%, or up to 80% or up to 90% essential soy phospholipids known as phosphatidylcholine with a pH of 7.2-7.6 but in no case higher than 8.2 and with DOC at 4.2% but preferably no greater than 4.6%. The compounded pharmaceutical is preferred cold-processed.

Option 2: (3-sn-Phosphatidyl)-cholin for subcutaneous and/or intracutanous use derived from soya based unsaturated alphalinolic acids (70% phosphatidylcholine), preferred but not restricted to the following preparation: 93-98% phospolipid with a preferrable DOC of 4.2% but no greater than 4.6%, Phenylmethanol of 3%, ethanol 95-97% (0.3-0.6%), natriumchloride, saline, 7-Natriumchlorid, Natriumhydroxid, DL-alpha-Tocopherol.

Option 3: The compound of option 1 with the composition (3-sn-Phosphatidyl)-cholin intended for subcutaneous and/or intracutanous use can also contain, preferred but not restricted to the following preparation: Polyenylphosphatidylcholinum derived from soya based unsaturated alphalinolic acids, with DOC 4.2% but preferrably no greater than 4.6%, ethanol 96% (0.3-0.6%), Pyridoxine, Cyanocobalamin, Nicotinic Acid, Pantothenic Acid, alpha-Tocopherolum, Sodium Chloride, Phenylmethanol of 3%, Sterile Water.

Option 4: The compound of option 1 with the composition (3-sn-Phosphatidyl)-cholin intended for subcutaneous and/or intracutanous use can also contain, preferred but not restricted to the following preparation: phosphatidylcholin derived from soya based unsaturated alphalinolic acids, with DOC 4.2% but no greater than 4.6%, ethanol 96% (0.3-0.6%), Sodium Chloride, Phenylmethanol of 3%, Sterile Water.

Option 5: The compound of option 1 with the composition (3-sn-Phosphatidyl)-cholin intended for subcutaneous and/or intracutanous use can also contain, preferred but not restricted to the following preparation as in sample of 50 ml: 70% phosphatidylcholin 2500 mg derived from soya based unsaturated alphalinolic acids, with 1250 mg DOC 4.2% but no greater than 4.6%, ethanol 120 mg (0.3-0.6%), 450 mg Benzyl alcohol, 10 mg Vitamin E and used with or without Sterile Water. pH can be 8.2, but preferably 7.2-7.6. The preparation is preferably used diluted by 50% NaCl to total PC volume, but not restricted to being diluted.

Option 6: The compound of option 1 with the composition (7, 8, 9, 10, and 11) of (3-sn-Phosphatidyl)-cholin intended for injectable cosmetic use can be used on its own, diluted, or in part or segment in various and multiple combinations to enhance cosmetic effect depending on the evaluation and composition of the fatty deposit with other compounds although not restricted to these compounds suggested as: 4-(1-Pyrrolidyl)-1-(2,4,6-Trimethoxyphenyl)-1-Butanone; 1H-Purine-2,6-dione, 3,7-dihydro-3,7-dimethyl-1-(5-oxohexyl)-; BL 191; a-Tocopherol; 2-methyl-3-all-trans-farnesyl digeranyl-1,4-naphthoquinone; Phylloquinone; B2; Cis-9, trans-11/Trans-10, cis 12 and diluted with NaCl.

Option 7: The compound of option 1 wherein the composition of phosphatidylcholine can further be used with the addition of CLA: Cis-9, trans-11 and Trans-10, cis 12;

Option 8: The compound of option 1 wherein the composition of phosphatidylcholine can further be used with the addition of K2: menaquinone: 2-methyl-3-all-trans-famesyl digeranyl-1,4-naphthoquinone;

Option 9: The compound of option 1 wherein the composition of phosphatidylcholine can further be used with the addition of K1: Phylloquinone and dihydrophylloquinone.

The Method of the Invention

A method of decreasing excess fat deposits in a human subject comprising administering to a human subject a lipolysis stimulating compound wherein said therapeutic composition can be administered transdermally.

The method of the invention wherein said therapeutic composition can be administered subcutaneously.

The method and compound of the invention, wherein said therapeutic composition can be administered intracutaneously.

The method and compound of the invention comprehends that the therapeutic composition can be administered as described in each individual option 1-6, or in combination with part or all of each option 1-6, or with the addition with options 7-9, either in part or separately in combination with phosphatidylcholine.

The method and compound of the invention wherein cosmetic reduction of adipose tissue (excess fat pads) can be achieved in areas, but not excluded to, fat deposits of the upper torso, lower torso including legs, feet, calves, hips and thighs; the face including eye fat pads; but not excluded to other cosmetic improvements on conditions that include skin retraction including medical conditions as lipomas, cellulite, buffalo hump, gynecomastia, excess mon pubis fat and other conditions caused by excess adipose appearance or metabolic disorders causing the increase of fat.

The method of the invention wherein said compound in full, diluted or adjusted part thereof is administered in a total dose of from about 1 ml to about maximum 3000 mg per fat deposit at any one given time to said human subject depending on condition and size of fat area.

The method of the invention wherein said decrease in body fat in said human subject can be measured within at least about eight weeks after administering the compound and may require several sessions for such cosmetic effect to take place.

1. A compound for decreasing excess fat deposits in a human subject comprising administering to a human subject a lipolysis stimulating compound selected from the group consisting of:
(3-sn-Phosphatidyl)-cholin for subcutaneous and/or intracutanous use derived from soya based unsaturated alphalinolic acids, preferably diluted but not restricted to being diluted for subcutaneous use in full, by its own, in part or mixed with other compounds although not restricted to any one, wherein the phosphatidylcholine used for subcutaneous use can contain 70%, or up to 80% or up to 90% essential soy phospholipids known as phosphatidylcholine preferably with a pH of 7.2-7.6 but never higher than 8.2 and with DOC at 4.2% but preferably no greater than 4.6%, and the compounded pharmaceutical is preferrably cold-processed.

2. The compound of claim 1, further wherein the (3-sn-Phosphatidyl)-cholin for subcutaneous and/or intracutanous use derived from soya based unsaturated alphalinolic acids (70% phosphatidylcholine), are preferably but not restricted to the following preparation: 93-98% phospolipid with a preferrable DOC of 4.2% but no greater than 4.6%, Phenylmethanol of 3%, ethanol 95-97% (0.3-0.6%), natriumchloride, saline, 7-Natriumchlorid, Natriumhydroxid, DL-alpha-Tocopherol.

3. The compound of claim 1, further wherein the composition (3-sn-Phosphatidyl)-cholin intended for subcutaneous and/or intracutanous use can also contain, preferably but not restricted to the following preparation: Polyenylphosphatidylcholinum derived from soya based unsaturated alphalinolic acids, with DOC 4.2% but preferrably no greater than 4.6%, ethanol 96% (0.3-0.6%), Pyridoxine, Cyanocobalamin, Nicotinic Acid, Pantothenic Acid, alpha-Tocopherolum, Sodium Chloride, Phenylmethanol of 3%, Sterile Water.

4. The compound of claim 1, further wherein the composition (3-sn-Phosphatidyl)-cholin intended for subcutaneous and/or intracutanous use can also contain, preferably but not restricted to the following preparation: phosphatidylcholin derived from soya based unsaturated alphalinolic acids, with DOC 4.2% but no greater than 4.6%, ethanol 96% (0.3-0.6%), Sodium Chloride, Phenylmethanol of 3%, Sterile Water.

5. The compound of claim 1, further wherein the composition (3-sn-Phosphatidyl)-cholin intended for subcutaneous and/or intracutanous use can also contain, preferably but not restricted to the following preparation as in sample of 50 ml: 70% phosphatidylcholin 2500 mg derived from soya based unsaturated alphalinolic acids, with 1250 mg DOC 4.2% but no greater than 4.6%, ethanol 120 mg (0.3-0.6%), 450 mg Benzyl alcohol, 10 mg Vitamin E and used with or without Sterile Water. pH can be 8.2, but preferably 7.2-7.6, and the preparation is preferably used diluted by 50% NaCl to total PC volume, but is not restricted to being used diluted.

6. The compound of claim 1, further wherein the composition of (3-sn-Phosphatidyl)-cholin intended for injectable cosmetic use can be used on its own, diluted, or in part or segment in various and multiple combinations to enhance cosmetic effect depending on the evaluation and composition of the fatty deposit with other compounds although not restricted to these compounds suggested as: 4-(1-Pyrrolidyl)-1-(2,4,6-Trimethoxyphenyl)-1-Butanone; 1H-Purine-2,6-dione, 3,7-dihydro-3,7-dimethyl-1-(5-oxohexyl)-; BL 191; a-Tocopherol; 2-methyl-3-all-trans-farnesyl digeranyl-1,4-naphthoquinone; Phylloquinone; B2; Cis-9, trans-11/Trans-10, cis 12 and diluted with NaCl.

7. The compound of claim 1, further wherein the composition of phosphatidylcholine can further be used with the addition of CLA: Cis-9, trans-11 and Trans-10, cis 12.

8. The compound of claim 1, further wherein the composition of phosphatidylcholine can further be used with the addition of K2: menaquinone: 2-methyl-3-all-trans-famesyl digeranyl-1,4-naphthoquinone.

9. The compound of claim 1, further wherein the composition of phosphatidylcholine can further be used with the addition of K1: Phylloquinone and dihydrophylloquinone.

10. A method for decreasing excess fat deposits in a human subject comprising administering to a human subject a lipolysis stimulating compound in accordance with claim 1, wherein said therapeutic composition is administered transdermally.

11. The method of claim 10, wherein said therapeutic composition can be administered subcutaneously.

12. The method of claim 10, wherein said therapeutic composition can be administered intracutaneously.

13. The method of claim 10, wherein said therapeutic composition administered can be selected from the compounds defined in claims 1-6 or in combination with or in addition to the compounds claimed in claims 7-9, either in part or separately in combination with phosphatidylcholine.

14. The method of claim 10, wherein said compound in full, diluted or adjusted part thereof is administered in a total dose of from about 1 ml to about 4000 mg per fat deposit at any one given time of said human subject depending on condition and size of fat area, but is not necessarily restricted to this amount.

15. The method of claim 10, wherein said decrease in body fat in said human subject can be measured within at least about eight weeks of said step of administering said compound and may require several sessions for such cosmetic effect to take place.
